(19) United States
(12) Patent Application Publication
(10) Pub. No.: US 2005/0032759 A1
(43) Pub. Date: Feb. 10, 2005

(54) Antitumor combined therapy

(76) Inventors: Giorgio Massimini, Milan (IT); Gabriella Piscitelli, Milan (IT); Giovanni Minardi, Milan (IT)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/333,384

(22) PCT Filed: Jul. 4, 2001
(86) PCT No.: PCT/EP01/07676

(30) Foreign Application Priority Data
Jul. 18, 2000 (GB) 0017635.4

(51) Int. Cl. A61K 39/395; A61K 31/58; A61K 31/56

(52) U.S. Cl. 514/176; 514/341; 514/182; 424/144.1; 514/397; 514/383; 514/18

(57) ABSTRACT

A method of treating a human being suffering from a hormone-dependent disorder characterized by the overexpression of EGFR, comprising administering to said human being an aromatase inhibitor and an EGFR antagonist or EGFR inhibitor, in amounts effective to produce a superadditive or synergistic therapeutic effect.

The present invention concerns the treatment of estrogen dependent disorders characterized by the overexpression of EGFR. More specifically, the invention concerns the treatment of a human being susceptible to or diagnosed with a disorder characterized by the overexpression of EGFR with a combination of an EGFR antagonist or inhibitor and an aromatase inhibitor.

The utility of aromatase inhibitors; EGFR antagonists and EGF inhibitors is well acknowledged in anticancer therapy. However, it is also well known in the art that administration to a patient of therapeutically effective amounts of aromatase inhibitors can cause considerable side effects. The major toxicities are for instance lethargy, hot flashes, rash, transient leukopenia, dizzines, nausea, constipation and vomiting. On the other hand, also administration to a patient of therapeutically effective amounts of an EGFR antagonist or inhibitor can similarly cause considerable side effects, e.g. hypersensitivity, alterations of renal function, myocardial lesions and cardiotoxicity in general.

The inventors of the present invention have found that a combination therapy of an hormone, in particular estrogen, dependent disorder characterized by the overexpression of EGFR, comprising a therapeutically effective amount of an aromatase inhibitor and a therapeutically effective amount of an EGFR antagonist or inhibitor, can produce a therapeutic effect which is-greater than that obtainable by single administration of a therapeutically effective amount of either a sole aromatase inhibitor or a sole EGFR antagonist or inhibitor.

Similarly they have found that a combination therapy of an hormone, in particular estrogen, dependent disorder characterized by the overexpression of EGFR, comprising a therapeutically sub-effective amount of an aromatase inhibitor and a therapeutically sub-effective amount of an EGFR antagonist or inhibitor, can produce substantially the same therapeutic effect, which is obtainable by single administration of a therapeutically effective amount of either an aromatase inhibitor or an EGFR antagonist or inhibitor. The most important, they have found that such newly obtained therapeutic effect is not paralleled by the toxic effects, otherwise caused by single administrations of either therapeutically effective amounts of an aromatase inhibitor or therapeutically effective amounts of an EGFR antagonist or inhibitor.

In view of the above, the effectiveness of an aromatase inhibitor and an EGFR antagonist or inhibitor is significantly increased without a parallel increased toxicity. In other words, the combined therapy of the present invention enhances the therapeutic effects of the aromatase inhibitor and the EGFR antagonist or inhibitor and thus yields more effective and less toxic treatment for hormone-dependent disorders.

Accordingly, the present invention provides a new and valuable tool in the therapy of hormone dependent disorders characterized by the overexpression of EGFR. The advantages provided by the present invention can be appreciated by their preferred features, described herebelow.

Examples of such disorders are cancers, e.g. breast, cervical, ovarian and endometrial cancers. However a preferred example of such disorders is breast cancer in a human being, in particular a female.

Accordingly, the present invention provides, as a first object, a pharmaceutical composition comprising an aromatase inhibitor and an EGFR antagonist or inhibitor, having a synergistic or superadditive therapeutic activity against an hormone-dependent disorder characterized by the overexpression of EGFR.

The present invention also provides the use of an aromatase inhibitor in the manufacture of a pharmaceutical composition for treatment of an hormone-dependent disorder characterized by the overexpression of EGFR, the treatment additionally comprising the administration of a composition comprising an EGFR antagonist or inhibitor, in amounts effective to produce a superadditive effect.

Examples of aromatase inhibitors according to the invention are exemestane, aminoglutethimide, roglethimide, pyridoglutethimide, anastrazole, trilostane, testolactone, formestane, atamestane, 1-methyl-1,4-androstadiene-3,17-dione (MAD), ketokonazole, fadrozole, letrozole, vorozole and anastrozole.

Preferred examples of aromatase inhibitors according to the invention are exemestane, anastrozole and letrozole, in particular exemestane.

The aromatase inhibitors cited herein are well known products, which are cited for instance in Cancer-Treat-Res.: 94, 231-254, 1998 and WO 99/30708.

An EGFR inhibitor is for instance compound ZM 105180, compound CP 358774 or compound ZD 1839. Compound ZM 105180 is 6-amino-4-(3-methylphenyl-amino)-quinazoline which is known from WO 95/03283. Compound CP 358774 is N-(3-ethylphenyl)-6,7-bis(2-methoxyethoxy)-4-quinazolinamine, which is known from WO 96/30347. Compound ZD 1839 is N(3-chloro-4-fluorophenyl)-7-methoxy-6-[3-(4-morpholinyl)-propoxy]4-quinazolinamine, which is known from WO 96/33980. Alternatively, an EGFR antagonist is for instance an antibody.

An antibody against EGFR, according to the invention, can be either an “intact” antibody or a fragment thereof.

The term “antibody” is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity. “Antibody fragments” comprise a portion of an intact antibody, preferably the antigen binding or variable region of the intact antibody. Examples of antibody fragments include Fab, Fab′, F(ab′)2, and Fv fragments; diabodies; linear antibodies; single-chain antbody molecules; and multispecific antibodies formed from antibody fragments.

An antibody against EGFR is in particular chimerized antibody C225 (cetuximab) and human antibodies E1.1, E2.4, E2.5, E6.2, E6.4, E2.11, E6.3 and E7.6.3, in particular E7.6.3. Preferred antibodies against EGFR are chimerized antibody C225 and human antibody E7.6.3. Chimerized antibody C225 is disclosed by WO 94/49210. Human antibodies E1.1, E2.4, E2.5, E6.2, E6.4, E2.11, E6.3 and E7.6.3 are disclosed by WO98/50433.

The present invention also provides a product comprising an aromatase inhibitor and an EGER antagonist or inhibitor, as combined preparation for simultaneous, separate or sequential administration, in amounts to produce a synergistic or superadditive therapeutic activity against an hormone-dependent disorder characterized by the overexpression of EGFR.

In a further aspect, the present invention provides a kit comprising, in a suitable container means, a pharmaceutical composition containing an aromatase inhibitor, as an active agent, and an EGFR antagonist or inhibitor, as a further active agent, in amounts to produce a synergistic or superadditive therapeutic activity against hormone-dependent disorder characterized by the overexpression of EGFR.

A further aspect of the present invention is to provide a method of treating a human being, particularly a female, suffering from an hormone-dependent disorder characterized by the overexpression of EGFR comprising administering to said human being an aromatase inhibitor and an EGFR antagonist or inhibitor, in amounts effective to produce a superadditive or synergistic therapeutic effect.

A still further aspect of the present invention is to provide a method for lowering the side effects (adverse reactions) caused by antitumor therapy with an aromatase inhibitor in a human being, particularly a female, suffering from an hormone-dependent tumor overexpressing EGFR, the method comprising administering to said human being a combined preparation comprising an aromatase inhibitor and an EGFR antagonist or inhibitor, in amounts effective to produce a superadditive or synergistic antitumor effect, while controlling the growth of neoplasm formation.

A still further aspect of the present invention is to provide a method for lowering the side effects (adverse reactions) caused by antitumor therapy with an EGFR antagonist or inhibitor in a human being, particularly a female, suffering from an hormone-dependent tumor overexpressing EGFR, the method comprising administering to said human being a combined preparation comprising an EGFR antagonist or inhibitor and an aromatase inhibitor, in amounts effective to produce a superadditive or synergistic antitumor effect, while controlling the growth of neoplasm formation.

The combination preparation according to the invention can also include combination packs or compositions in which the constituents are placed side by side and can be administered simultaneously, separately or sequentially to one and the same human being. Accordingly, the aromatase inhibitor and the EGFR antagonist or inhibitor may be present within a single or distinct container.

By the term “a superadditive or synergistic antitumor effect” as used herein is meant the inhibition of the growth tumor, preferably the complete regression of the tumor, by administering a combination of an aromatase inhibitor, as defined above, and an EGFR antagonist or inhibitor, to a human being, particularly a human female.

Said preparation having therefore a potentiated antitumor (superadditive) activity with respect to products containing either an aromatase inhibitor or an EGFR antagonist or inhibitor, which is greater than the sum of the actions of individual components.

By the term “administered” or “administering” as used herein is meant any acceptable manner of administering a drug to a patient which is medically acceptable including parenteral and oral administration. Preferably the aromatase inhibitor and the EGFR antagonist or inhibitor are administered in a sequential, separate or substantially simultaneous manner.

By “parenteral” is meant intravenous, subcutaneous, intradermal or intramuscular administration.

Oral administration includes administering the constituents of the combined preparation in a suitable oral form such as, e.g., tablets, capsules, suspensions, solutions, emulsions, powders, syrups and the like.

Parenteral administration includes administering the constituents of the combined preparation by subcutaneous, subcutaneous, intravenous or intramuscular injections.

The actual preferred method and order of administration of the combined preparations of the invention may vary according to, inter alia, the particular pharmaceutical formulation of the aromatase inhibitor being utilized, the particular pharmaceutical formulation of the EGER antagonist or inhibitor being utilized, the particular cancer being treated and the particular patient being treated.

The dosage ranges for the administration of the combined preparation may vary with the age, condition and extent of the disease in the patient and can be determined by one of skill in the art.

The dosage regimen must therefore be tailored to the particular of the patient's conditions, response and associate treatments in a manner which is conventional for any therapy, and may need to be adjusted in response to changes in conditions and/or in light of other clinical conditions.

In the combined method of treatment according to the subject invention, the aromatase inhibitor may be administered simultaneously with the EGFR antagonist or inhibitor or the compounds may be administered sequentially, in either order.

An effective amount of an aromatase inhibitor antitumor agent may vary from about 0.5 to about 500 mg pro dose 1-2 times a day. Exemestane, for example, may be administered orally in a dosage range varying from about 5 to about 200 mg, and particularly, from about 10 to about 25 mg, or parenterally from about 50 to about 500 mg, in particular from about 100 to about 250 mg. Fadrozole, for example, may be administered orally in a dosage range varying from about 0.5 to about 10 mg, and particularly, from about 1 to about 2 mg.

Letrozole, for example, may be administered orally in a dosage range varying from about 0.5 to about 10 mg, and particularly, from about 1 to about 2.5 mg.

Formestane, for example, may be administered parenterally in a dosage range varyirig from about 250 to about 500 mg, and particularly, from about 250 to about 300 mg.

Anastrozole, for example, may be administered orally in a dosage range varying from about 0.5 to about 10 mg, and particularly, from about 1 to about 2 mg.

In the method of the subject invention, for example for the administration of the recombinant humanized monoclonal antibody anti-EGFR C225 (cetuximab), the course of therapy generally employed is from about 150 to about 500 mg/m² of body surface area. Preferably, the course therapy employed consists of a loading dose of about 400 mg/m², followed by weekly maintenance dosage of about 180-250 mg/m². According to a preferred embodiment of the invention patients are given an injection of cetuximab as a weekly, dose escalating 4-week protocol, with doses up to 200 mg/m². If the disease is stabilized, then a further 8-week course can begin.

In the method of the subject invention, for the administration e.g. of the recombinant humanized monoclonal antibody E7.6.3 the course of therapy generally employed is from about 1 to about 1000 mg/m² of body surface area. More preferably, the course therapy employed is from about 60 to about 600 mg/m² of body surface area.

In the method of the subject invention, for the administration e.g. of compound CP-358774 the course of therapy generally employed is from about 25 to about 150 mg/day p.os., so that to reach a plasma concentration from about 300 to about 700 ng/ml, preferably 500 ng/ml.

In the method of the subject invention, for the administration e.g. of compound ZD 1839 the course of therapy generally employed is from about 50 to about 300 mg/day p.os.

The therapy method according to the present invention is, in particular, suitable for treating a human being suffering from hormone dependent disorders, characterized by the overexpression of EGFR. Typical examples of such disorders are tumors, like ovarian, cervical and endometrial cancers in a human female or breast cancer in a human being, in particular a female.

More in particular, the combined use of an aromatase inhibitor, according to the invention, preferably exemestane, and an EGFR antagonist, for example antibody C225 or E7.6.3 or an EGFR inhibitor, e.g. compounds CP-358774, ZD 1839 or ZM 105180, can be suitable for the treatment of patients with cancers over-expressing EGFR, for example, for patient with breast cancer, in particular with metastatic breast cancer, over-expressing EGFR.

According to a preferred aspect of the present invention the superadditive antitumor effect results in an anti breast cancer therapy having increased effectiveness in controlling, i.e. slowing, interrupting, arresting, stopping or reversing, the neoplasm formation.

As used herein, “controlling the growth” of the neoplasm refers to slowing, interrupting, arresting or stopping its growth and it does not necessarily indicate a total elimination of the neoplasm.

Therefore, the term “treating” simply means that the life expectancy of an individual affected with a cancer will be increased, that one or more of the symptoms of the disease will be reduced and/or that quality of life will be enhanced.

The compositions and combined therapy method of the invention provide therefore a two-way attack in particular on cancer cell growth. Exemestane in view of its biological properties, is the most preferred example of aromatase inhibitor according to the invention.

From the pharmacological point of view, the valuable biological properties of exemestane may be found in its peculiar mechanism of aromatase inactivation.

The aromatase enzyme (450ₐᵣₒₘ) is a specific form of cytochrome P450 hemoprotein composed of a P450 (heme) moiety and a peptidic moiety. The enzyme catalyzes a multistep reaction leading to aromatization of the A ring of the androgen substrate (mainly androstenedione) to estrone, requiring the presence of the cofactor NADPH.

After this enzymatic reactions the enzyme molecule is once more available to perform a new aromatization.

The exemestane's mechanism of aromatase inhibition has been extensively studied and the compound has been found to cause enzyme inactivation. In fact exemestane, structurally related to the natural substrate androstenedione, is initially recognized by the aromatase enzyme as a false substrate, therefore competes with androstenedione at the active site of the enzyme. The compound is then transformed (through and NADPH-dependent mechanism) to an intermediate which binds irreversibly to the enzyme causing its inactivation (also known as suicide inhibition). Therefore the enzyme is definitely inactivated and de novo enzyme synthesis is required for oestrogen production.

Suitable modifications and adaptations of a variety of conditions and parameters normally encountered in clinical therapy which are obvious to those skilled in the art are within the scope of this invention.

A pharmaceutically composition containing an aromatase inhibitor and/or an EGFR antagonist or inhibitor can be prepared according to well known techniques to those skilled in the art. For instance a pharmaceutical composition containing exemestane can be prepared according to U.S. Pat. No. 4,808,616.

1. Use of an aromatase inhibitor in the manufacture of a pharmaceutical composition for the treatment of a hormone-dependent disorder characterized by the overexpression of EGFR, wherein the pharmaceutical composition is for administration in combination therapy with a composition comprising an EGFR antagonist or an EGFR inhibitor, in amounts effective to produce a superadditive or synergistic effect.

2. Use, according to claim 1, wherein the disorder is breast, cervical, ovarian or endometrial cancer.

3. Use, according to claim 2, wherein the disorder is breast cancer.

4. Use, according to any one of. the preceding claims, wherein the aromatase inhibitor is selected from exemestane, aminoglutethimide, roglethimide, pyridoglutethimide, anastrazole, trilostane, testolactone, formestane, atamestane, 1-methyl-1,4-androstadiene-3,17-dione (MAD), ketokonazole, fadrozole, letrozole, vorozole and anastrozole.

5. Use, according to claim 4, wherein the aromatase inhibitor is exemestane.

6. Use, according to any one of the preceding claims, wherein the EGER inhibitor is an antibody against EGFR.

7. Use, according to claim 6, wherein the antibody is selected from chimerized antibody C225 and human antibodies E1.1, E2.4, E2.5, E6.2, E6.4, E2.11, E6.3 and E7.6.3.

8. Use according to claim 6, wherein the antibody is selected from chimerized antibody C225 and human antibody E7.6.3.

9. Use, according to any one of claims 1 to 5, wherein the EGFR antagonist is selected from ZM 105180,CP-358774 and ZD 1839.

10. A product comprising an aromatase inhibitor and an EGFR antagonist or EGFR inhibitor, as a combined preparation for simultaneous, separate or sequential administration, in amounts effective to produce a synergistic or superadditive therapeutic effect, in the treatment of a hormone-dependent disorder characterized by the overexpression of EGFR.

11. A method of treating a human being suffering from a hormone-dependent disorder characterized by the overexpression of EGFR, said method comprising administering to said human being, simultaneously, separately or sequentially, an aromatase inhibitor and an EGFR antagonist or EGFR inhibitor, in amounts effective to produce, a superadditive or synergistic therapeutic effect.

12. A method for reducing the side effects caused by the treatment of a human being suffering from a hormone-dependent tumor overexpressing EGFR, with an aromatase inhibitor, the method comprising administering to said human being a combined preparation comprising an aromatase inhibitor and an EGFR antagonist or an EGFR inhibitor, in amounts effective to produce a superadditive or synergistic antitumor effect, while controlling the growth of neoplasm formation.

13. A method for reducing the side effects caused by the treatment of a human being suffering from a hormone-dependent tumor overexpressing EGFR with an EGFR antagonist or EGFR inhibitor, the method comprising administering to said human being a combined preparation comprising an EGFR antagonist or an EGER inhibitor and an aromatase inhibitor, in amounts effective to produce a superadditive or synergistic antitumor effect, while controlling the growth of neoplasm formation.

14. A method according to any one. of claims 11, 12 or 13 wherein the aromatase inhibitor and the EGFR antagonist or EGFR inhibitor are administered in a sequential, separate or a substantially simultaneous manner.
